(19) United States
(12) Patent Application Publication
(10) Pub. No.: US 2017/0100326 A1
(43) Pub. Date: Apr. 13, 2017

(54) LIGHTENING ACTIVE AGENT CONTAINING PLANT EXTRACTS, USES THEREOF AND COMPOSITIONS CONTAINING THE SAME

(71) Applicant: CHEMYUNION QUÍMICA LTDA., Sorocaba (BR)

(72) Inventors: Cecília Nogueira, Sorocaba (BR); Marcos Roberto Rossan, Sorocaba (BR); Cristiane Rodrigues da Silva Pacheco, Sao Paulo (BR); Edson Katekawa, Sorocaba (BR); Renata Miliani Martinez, Sorocaba (BR); Camila Kappke Mariano Cesar, Sorocaba (BR); Wagner Vidal Magalhães, Sorocaba (BR)

(73) Assignee: CHEMYUNION QUÍMICA LTDA., Sorocaba (BR)

(21) Appl. No.: 15/313,037

(22) PCT Filed: May 21, 2014
(86) PCT No.: PCT/BR2014/000170

§ 371 Date: Nov. 21, 2016

(51) Int. Cl. A61K 8/97; A61Q 19/02; A61Q 17/04; A61K 8/891; A61K 8/37; A61K 8/67; A61K 8/34; A61K 8/73; A61K 8/55; A61K 8/36

(52) U.S. Cl. CPC A61K 8/97; A61K 8/553; A61Q 19/02; A61Q 17/04; A61K 8/36; A61K 8/37; A61K 8/678; A61K 8/34; A61K 8/73; A61K 8/891; A61K 2800/70

(57) ABSTRACT

The present invention discloses a cosmetic composition intended for skin whitening, more particularly a whitening agent comprising the extract of Alpinia officinarum or the association between extracts of Physalis angulata, Bidens pilosa and Achyrocline satureioides included in a structured lipid network for delivery of bioactive compounds.

### FIELD OF THE INVENTION

The present invention discloses a cosmetic composition intended for skin whitening, more particularly a whitening agent comprising the extract of Alpinia officinarum or the association between extracts of Physalis angulata, Bidens pilosa and Achyrocline satureioides included in a structured lipid network for delivery of bioactive compounds.

### BACKGROUND OF THE INVENTION

Skin hyperpigmentation is considered a usual dermatological response causing discomfort to individuals suffering from it, since it significantly affects psychological well-being, contributing to reductions in productivity, social activities and self-esteem. It is basically characterized by increased production and accumulation of melamine over the skin. As the main factors responsible for this change, we can mention endocrinal dysfunctions, exposure to solar radiation, pregnancy, genetic factors, ageing and skin inflammations caused by acne or contact dermatitis.

There are currently various treatments for skin pigmentation dysfunctions, but only a few really provide satisfactory efficacy and safety. As the main examples of whiteners in this class, we highlight hydroquinone, arbutin and kojic acid. Studies suggest that the topical application of hydroquinone, one of the most used whitening agents in the past, may disturb extracellular matrix fibers (especially collagen and elastin), causing changes in supra-renal glands and thyroid physiology, as well as loss in skin firmness.

Furthermore, despite the initial whitening effect, hydroquinone and arbutin may cause ochronosis, a condition wherein the skin becomes more pigmented than at the start of the treatment. On the other hand, kojic acid, an aromatic acid with fungal origin acting as a chelating agent, was considered as able to promote the development of cancer in the liver and thyroid in rats. Contact dermatitis, skin eruptions, burning sensation and increase in skin susceptibility for the effects of UV radiation are other usual effects attributed to the application of these classic pharmacological substances.

In this context, natural products and plant extracts with nontoxic properties and which are environmentally safe have great potential for the development of therapeutic agents for hyperpigmentation treatment, or even for application as cosmetic skin whitening formulations.

A few publications related to the use of extracts intended for cosmetic depigmentation application have been developed:

U.S. Pat. No. 5,980,904 discloses a composition for topical use containing glycol extract of bearberry obtained from Arctostaphylos uva-ursi in combination with one or more depigmenting agents.

U.S. Pat. No. 5,747,006 discloses the use of a composition for topical use containing fermented Malpighia glabra and a whitening agent, wherein the extract of fermented Malpighia glabra is substantially free from ascorbic acid.

Publication WO 2010/098533 discloses the de-pigmenting activity of the extract of Ecklonia cava, brown alga, and the compound 7-pholoroeckol, isolated from the alga itself, in a cosmetic and pharmaceutical composition.

The publication WO 2004/105718 discloses a cosmetic composition for topical application, prepared by the use of extracts of plants from the families Symplocos, Rubia or their combinations.

The publication WO 2010/004355 discloses a cosmetic composition containing extracts of Glycyrrhiza glabra, Valeria indica, Hedychium spicatum, Alpinia galanga and their combinations, and pharmaceutically/cosmetically acceptable excipients. All the extracts were obtained by extraction using organic solvents such as chloroform, acetone, methanol and n-hexane.

The document JP2010100563 discloses the preparation of a depigmenting agent from one or more almonds selected from species Prunus armeniaca, Prunus mume, Prunus persica, Eriobottrya japonica and Alpinia galanga.

The document JP2010189312 discloses the use of extracts of Alpinia galangal and Daphne tangutica or their combinations, intended for the cosmetic application of depigmentation.

None of the documents as mentioned above discloses cosmetic compositions containing the extracts, severely or in association, as used in the present invention.

Matsuda et al (Bioorg. Med. Chem. 17, 2009, 6048-6053) disclose the effect of extracts of Alpinia officinarum (Galanga Pequena), obtained in a solution of 80% (v/v) acetone and in ethyl acetate solution over melanogenesis. The effect as disclosed is restricted to the reduction of melamine synthesis in 4A5 cells from murine melanoma. Similarly, Lu et al (J. Enzyme Inhib. Med. Chem. 22, 2007, 433-438) disclose the reduction of the pigment synthesis by 21% in an in vitro assay from the treatment with a mixture of flavonoids, isolated from Alpinia officinarum by means of different extracting solvents, including petroleum ether and ethyl acetate.

It is known that the use of organic solvents, such as ethyl acetate, petroleum ether and acetone, for the preparation of plant extracts has restricted cosmetic application due to toxic effects and the ability to promote dermatitis while in contact with the skin.

Furthermore, plant extracts have characteristics making their incorporation into finished cosmetic products become difficult, such as color, odor, low stability and others. The color parameter is, in the case of whiteners, one of the largest limiting factors to the use of plant extracts. Besides depigmenting efficacy, whitening products require white appearance, or a very subtle color.

In case of extracts of Alpinia officinarum, a strong reddish brown color is a unique characteristic.

The U.S. Pat. No. 8,101,211 discloses the inhibiting ability for melamine formation of extracts of Bidens pilosa and Physalis angulata, isolated. However, the publication as mentioned does not report the whitening ability from ex vivo or in vivo assays evaluating its efficacy in human beings, nor does it disclose any synergism from the combinations of the extracts as mentioned.

The object of the present invention is to develop an active skin whitening ingredient overcoming adversities and limitations in the state of the art. It was of special interest to obtain a highly effective and safe whitening active agent, which could be easily incorporated into cosmetic compositions with no contribution to the color of the final formulation, and which required concentration would not interfere in the formulation of the cosmetic composition, being thus highly effective under low concentrations.

We have noticed that the active whitening agent, object of the present invention, may be incorporated at highly effective amounts in cosmetic compositions. Furthermore, the active ingredient promotes small changes in color and, in some cases, this parameter does not change. Additionally, the whitening active ingredient of the invention does not develop any irritation on skin.

Whitening active ingredients prepared according to the invention allow the formulation of highly effective cosmetic compositions, promoting small changes in the color of cosmetic compositions, and are safe for topical application.

### BRIEF DESCRIPTION OF THE INVENTION

The present invention discloses a whitening active agent comprising:

(a) extract of Alpinia officinarum (a-1) or the association of extracts of Physalis angulata (a-2), Bidens pilosa (a-3) and Achyrocline satureioides (a-4);

(b) structured lipid network for delivery of bioactive compounds.

The extract of Alpinia officinarum as provided by that delivery system, even under a 20-time lower concentration, has higher whitening efficacy than the same extract in free form. Said characteristic enables the incorporation of the active principle into cosmetic compositions, not promoting changes in color.

Similarly, the association of extracts of Physalis angulata, Bidens pilosa and Achyrocline satureioides promotes higher depigmentation effect than that offered by isolated extracts, causing an unexpected benefit effect, the synergetic effect of the combination.

The invention also contemplates a skin whitening process comprising the topic application of the cosmetic composition of the invention over the skin.

The invention also contemplates a process for treatment or prevention of pigmenting dysfunctions, comprising the topic application of the cosmetic composition of the invention over the skin.

Furthermore, the invention contemplates the use of a whitening active ingredient as defined by the present application for the production of a composition for skin whitening.

Additionally, the use of the cosmetic composition of the invention for skin whitening is claimed.

Furthermore, a process for the manufacture of the whitening active ingredient of the present invention by mixing the extract of Alpinia officinarum (a-1) or associating extracts of Physalis angulata (a-2), Bidens pilosa (a-3) and Achyrocline satureioides (a-4) in a structured lipid network, obtained by means of a lipid agent in the presence of an ethanol phase for the delivery of bioactive compounds is also contemplated.

Finally, an association comprising extracts of Physalis angulata, Bidens pilosa and Achyroclline satureioides is also claimed.

### BRIEF DESCRIPTION OF THE DRAWINGS

FIG. 1 shows the results of the melanogenesis inhibition assay ex vivo.

FIG. 2 shows the result of the incorporation of the extract of Alpinia officinarum in the free form of the whitening active ingredient containing the extract of Alpinia officinarum and the structured lipid network in a cosmetic composition.

### DETAILED DESCRIPTION OF THE INVENTION

The term “whitening active ingredient” as used throughout the invention covers any cosmetic application which object is to change the color or pigmentation of the skin to a clearer color or shade in comparison with the state before the treatment with the active whitening agent. Therefore, the use of the whitening active ingredient covers general skin whitening, removing or clearing spots, lentigo, melasma or other forms of hyperchromia, which may be caused by excessive sun exposure, hormone unbalance, use of medicines, pregnancy, inflammatory processes or genetic predisposition.

The term “whitening active agent” as used throughout the invention also covers any application which object is to avoid or prevent skin pigmentation into a darker color or shade in comparison with the original shade. Said application is provided as an example by the use in sunscreens.

Component (A-1)—Extract of Alpinia officinarum

The whitening active agent of the invention may comprise the extract of Alpinia officinarum. A member of the family Zingiberaceae, Alpinia officinarum (usually known as Galanga Pequena) is used for seasoning and in the traditional Chinese medicine for various purposes, such as stomachic and carminative.

Any or all parts of Alpinia officinarum may be used to prepare the extract of the invention. The plant or parts of the plant, fresh or dried, may be mechanically processed so to reduce their size. Preferably, dry rhizomae of Alpinia officinarum, ground or milled, are used.

Constituent (A-2)—Extract of Physalis angulata

A member of the family Solanaceae, Physalis angulata (usually known as juazeiro), usually grows as a weed in the Northern and Northeastern Regions in Brazil, but is also found in other tropical regions in Africa, Americas and Asia. In Brazil, it has been used in popular medicine, as an anti-rheumatic and diuretic, hepatoprotector, anti-inflammatory, anti-cough, analgesic and anti-malaria agent, and in jaundice cases.

Any or all parts of Physalis angulata may be used to prepare the extract of the invention. The plant or parts of the plant, fresh or dried, may be mechanically processed so to reduce their size. Preferably, dry leaves, stems and roots of Physalis angulata, be them ground or milled, are used.

Component (A-3)—Extract of Bidens pilosa

A member of the family Asteraceae, Bidens pilosa (usually known as beggar's tick) is usually employed for the treatment of foot-and-mouth disease, angina, diabetes, menstrual disorders, various kinds of hepatitis, laryngitis, intestinal constipation and dermatological and internal inflammatory processes. Furthermore, it is usually employed in the Peruvian phytotherapeutic medicine as an adjuvant for the treatment of hepatitis, conjunctivitis, abscesses, fungal infections and to prevent sudden loss of weight in children.

Any or all parts of Bidens pilosa may be used to prepare the extract of the invention. The plant or parts of the plant, fresh or dried, may be mechanically processed so to reduce their size. Preferably, dry leaves, stems and roots of Bidens pilosa, be them ground or milled, are used.

Component (A-4)—Extract of Achyrocline satureioides

A member of the family Ateraceae, Achyrocline satureioides, is used in the Brazilian popular medicine as an anti-inflammatory, hypoglycemic, digestive, anti-spasmodic agent and for the treatment of gastrointestinal disorders.

Any or all parts of Achyrocline satureioides may be used to prepare the extract of the invention. The plant or parts of the plant, fresh or dried, may be mechanically processed so to reduce their size. Preferably, dry flowers of Achyrocline satureioides, be them ground or milled, are used.

The components a-1, a-2, a-3 and a-4 of the invention may be prepared by means of well-known extraction methods. Said methods include processes of maceration, decoction, digestion, re-maceration, ultrasonic extraction, extraction using supercritical fluid or solid-liquid extraction under continued reflux in a Soxhlet extractor. Extraction with solvents like ethanol, glycols and their mixtures, be them in the presence of water or not, are preferably used. As a preferable application, the extract is obtained by an extracting process with the solvents water and butylene glycol, under the ratio 60:40. The extracts of the invention may also include usual conservation agents in the art, such as phenoxyethanol and potassium sorbate.

The extraction process is usually performed under shaking and at a temperature between 4 and 100° C., preferably between 35 and 65° C. for a period between 1 and 12 hours, preferably between 1 and 5 hours.

Component B—Delivery System

The delivery system is a composition containing transdermal release promoters. Transdermal release promoters are chemical compounds, usually with amphiphilic character, which may permeate or interact with the constituents of the stratum corneum. Such permeation is made by the reversible change of function of the skin barrier, which occurs by changing the usual order of intercell lipids in the presence of the permeation promoter.

The delivery system comprises at least a lipid agent selected from the group including: squalene, lecithin, phosphatidylcholine (originating from soy or egg), cholesterol, L-a-dioleoyl phosphatidylethanolamine, dimethyldioctadecyl ammonium bromide, 1,2-dioleoyl-3-trimethylammonium propane, 1,2-diacyl-3-dimethylammonium propane, dioleoxypropyltrimethylammonium, 2,3-dioleoyloxy-N-[(sperminocarboxamine)ethyl]-N,N-dimethyl-1-propanamine], dioctadecyl dimethylammonium bromide, dimiristoylphosphatidylcholine, distearoylphosphatidylcholine, dilauroylphosphatidylcholine, dipalmitoylphosphatidylcholine, phosphatidylethanolamine, phosphatidylinositol, sphingomyelin, ceramides and linoleic acid. As a preferable application, the delivery system is prepared with soy phosphatidylcholine.

The delivery system of the invention also comprises at least one hydrocolloid selected from the group including, with no restrictions, cellulose and derivatives (e. g. hydroxyethylcellulose, carboxymethylcellulose, methylcellulose), gum Arabic, karaya gum, ghatti gum, tragacanth gum, starch and derivatives, pectin (esterified or starched), guar gum, locust bean gum, tara gum, tamarind gum, konjac manana, agar, carrageen, alginate, xanthan gum, curdlan, dextran, gellan gum, gelatin, caseinate, milk serum protein and chitosan.

The method used to prepare the lipid network as used in the present invention was the injection of adapted ethanol, by which the lipid agent has been solubilized in an ethanol phase and added over the water phase containing plant extracts and preservatives. After full addition of the ethanol phase, the medium was maintained under shaking and heated with the addition of xanthan gum and vegetal glycerin.

The invention also comprises a cosmetic composition containing the whitening active ingredient of the invention. The cosmetic composition of the invention may comprise any cosmetically acceptable carriers and excipient and/or additives as known in the state of the art.

The whitening active agent may be included within the concentration range between 0.1 and 20.0% (w/v) of the composition, preferably between 0.1 and 10.0% (w/v).

The cosmetic composition of the invention may also comprise other constituents. In one of the embodiments of the invention, the composition comprises at least one representative of the group consisting of UV ray blocking agents, emulsifiers, emollients, thickeners and other whitening active ingredients.

The addition of organic blocking agents such as acrylates (e. g. ethyl 2-cyano 3,3-diphenylacrylate and 2-ethyl-hexyl 2-cyano-3,3-diphenylacrylate), benzophenones (e. g. benzofenone-3 and benzofenone-4); imidazole derivatives (e. g. 2-phenyl benzimidazole 5-sulfonic acid); p-aminobenzoic acid derivatives (e. g. p-aminobenzoic acid and glyceryl p-aminobenzoate); benzotriazole derivatives (e. g. methylene-bis-benzotriazolyl tetramethylbutylphenol), with no restrictions to these examples, favors the depigmentation process, since it restricts melanogenesis stimulation as induced by UV radiation.

Another class of appropriate blocking agents which may be incorporated into the cosmetic composition of the invention is that of inorganic pigments, such as titanium oxide, zinc oxide, iron oxide and zirconium oxide.

UV blocking agents may be included under a weight percentage between 0.5 and 10.0% of the composition, preferably between 1.0 and 7.0%.

The use of emulsifying agents such as sorbitol esters, polyglycerol esters and stearates (e. g. magnesium stearate and sodium stearate) allows for the homogeneous combination of immiscible constituents, besides acting in the stabilization of the composition. The emulsifier may be included under a weight percentage between 0.5 and 15.0% of the composition, preferably between 2.0 and 10.0%.

The incorporation of emollient agents into the composition such as mineral oils, cholesterol, lanolin, silicones, vegetal oils (such as almond oil, sunflower oil and coconut oil), fatty acids and fatty alcohols has the purpose to keep skin hydration, since these agents avoid water evaporation from the skin. The emollient may be included under a weight percentage between 0.5 and 10.0% of the composition, preferably between 0.5 and 7.0%.

The addition of thickening agents such as carbomer, vegetal waxes (e. g. bee wax), dioxides (e. g. silicon dioxide), gums (e. g. xanthan gum) and polyacrylamides helps to obtain an adequate consistence for the composition. The thickener may be included at a weight percentage between 0.1 and 20.0% of the composition, preferably between 1.0 and 7.0%.

The incorporation of antioxidizing agents such as tocopherol, tocopherol derivatives, ascorbic acid, BHT and BHA has the object to preserve constituents of the composition against oxidization. The antioxidant may be included under a weight percentage between 0.1 and 5.0% of the composition, preferably between 1.0 and 5.0%.

In an embodiment of the present invention, the simultaneous use of the whitening active agent of the invention with other whitening agents as known in the state of the art, such as kojic acid, alpha-arbutin, beta-arbutin, hydroquinone, linoleic acid, azelaic acid, ferulic acid, ascorbic acid, niacinamide, resveratrol, extracts of Morus alba and extracts of Glycyrrhiza glabra. The additional whitening agent may be included under a weight percentage between 0.001 and 10% of the composition, preferably between 0.1 and 3%.

The cosmetic composition of the invention is preferably used as a skin whitening agent or also as a melanogenesis inhibitor.

The cosmetic composition of the invention may be present in any galenic form as known in the state of the art, preferably in the form of creams, lotions, gels, ointments, emulsions and powders.

### EXAMPLES

Although the examples below provide a more detailed description of the invention, they are merely illustrative and the invention should not be limited to these.

### Example 1

### Preparation of Extract of Alpina officinarum

1.0 kg of dried and ground rhizome of Alpinia officinarum Hance was added to 5.03 kg of distilled water, 3.79 kg of butyleneglycol, 0.10 kg of phenoxyethanol and 0.08 kg of potassium sorbate and kept under heating at 40-45° C. and shaking for five hours. Subsequently, the extract was filtered through filter paper under vacuum, using celite. pH of the extract was corrected to 5.0-6.20 with 50% citric acid solution.

### Example 2

### Preparation of Extract of Physalis angulata

1.0 kg of ground leaves, stems and roots of Physalis angulata was added to 5.03 kg of distilled water, 3.79 kg of butyleneglycol, 0.10 kg of phenoxyethanol and 0.08 kg of potassium sorbate and kept under heating at 40-45° C. and shaking for five hours. Subsequently, the extract was filtered through filter paper under vacuum, using celite. pH of the extract was corrected to 5.0-6.20 with 50% citric acid solution.

### Example 3

### Preparation of Extract of Bidens pilosa

1.0 kg of ground leaves, stems and roots of Bidens pilosa was added to 5.03 kg of distilled water, 3.79 kg of butyleneglycol, 0.10 kg of phenoxyethanol and 0.08 kg of potassium sorbate and kept under heating at 40-45° C. and shaking for five hours. Subsequently, the extract was filtered through filter paper under vacuum, using celite. pH of the extract was corrected to 5.0-6.20 with 50% citric acid solution.

### Example 4

### Preparation of Extract of Achyrocline satureioides

1.0 kg of ground leaves, stems and roots of Achyrocline satureoides was added to 5.03 kg of distilled water, 3.79 kg of butyleneglycol, 0.10 kg of phenoxyethanol and 0.08 kg of potassium sorbate and kept under heating at 40-45° C. and shaking for five hours. Subsequently, the extract was filtered through filter paper under vacuum, using celite. pH of the extract was corrected to 5.0-6.20 with 50% citric acid solution.

### Example 5

### Preparation of a Composition Containing Extract of Alpinia officinarum Carried in a Delivery System

In the ethanol phase, 8.0 kg of 90% soy phosphatidylcholine were mixed with 15.00 kg of 96% neutral ethanol until full homogenization (600 rpm). In the water phase, 1.0 kg of phenoxyethanol and 0.6 kg of potassium sorbate were mixed at 69.2 kg of deionized water until full solubilization. Subsequently, 5.0 kg of extract of Alpinia officinarum (600 rpm) have been added under shaking for 10 minutes. After preparation of the ethanol phase, it has been continuously added to the water phase under shaking (600 rpm). After full addition of the ethanol phase, the product has been kept under shaking for 30 minutes. Finally, the product has been heated (40-45° C.) for the addition of the mixture formed by 0.40 kg of xanthan gum and 0.80 kg of vegetal glycerin. The product has been kept under shaking (600 rpm) and heating (40-45° C.) for one hour. At the end of the process, the product carried in a delivery system has been filtered through a 100 micron nylon filter.

### Example 6

### In Vitro Melanogenesis Inhibition Assay

Melanocytes (line B16) were cultivated for 24 hours. Culture media was then removed and substituted with new media containing each one of the ingredients to be assayed (under the respectively higher non-cytotoxic concentrations) or without any ingredient (control group). After 72 hours of incubation, melamine levels were measured from the determination of optical density at 475 nm with the support of a standard curve of melamine.

Combinations of ingredients were assayed at the same cell cultures, in parallel with individual ingredients. Results are expressed in % over the control group, from an average of three independent assays.

A comparative analysis of the quantity of melamine as produced by cell cultures treated with different extracts and their combination was performed. Table 1 shows that isolated extracts of Bidens pilosa, Physalis angulata and Achyrocline satureioides reduce melamine levels to 81%, 72% and 53%, respectively. Surprisingly, treatment with isolated extract of Alpinia officinarum carried in a delivery system resulted in levels of melamine equivalent to 30% over the control group. Similarly, the treatment using the combination of extracts of Bidens pilosa, Physalis angulata and Achyrocline satureioides officinarum, also carried in a delivery system, resulted in melamine levels equivalent to 39% over the control group.

TABLE 1

| | % Melanin over |
|---|---|
| Treatment | the control group |
| Control | 100 |
| Alpinia officinarum | 30 |
| Bidens pilosa | 81 |
| Physalis angulata | 72 |
| Achyrocline satureioides | 53 |
| Combination of Bidens pilosa, Physalis | 39 |
| angulata and Achyrocline satureioides | |

### Example 7

### Ex Vivo Melanogenesis Inhibition Assay

Fragments of human skin were obtained from blepharoplasties, cut into pieces of approximately 1 cm² and incubated with a cosmetic composition containing 3% free extract of Alpinia officinarum, 3% Alpinia officinarum in a delivery system, 2% arbutin or 2% kojic acid.

After treatment, skin samples were fixed over 4% (w/v) paraformaldehyde for 24 hours and cryoprotected in 30% sucrose solution for 48 hours. After a cryoprotection period, the materials were immersed in assembly media for the inclusion of cuts in cryostat, followed by 10 μm thick serial cuts with the help of a cryostat (LEICA-CM1850), which were extended over glass slides. Subsequently, cuts were colored by the Fontana Masson technique.

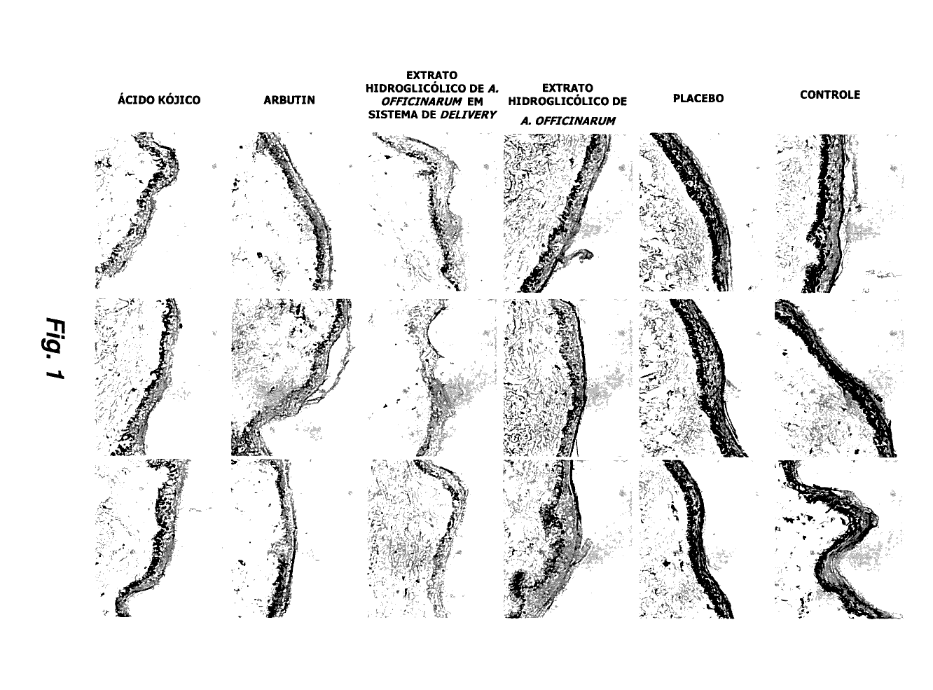

Results are available on FIG. 1 and show the depigmenting ability of the hydroglycol extract of Alpinia officinarum. Surprisingly, the effects of the same extract are potentialized when carried by the delivery system as disclosed by the invention. Even more surprisingly, the whitening effect as offered by the extract of Alpinia officinarum carried by a delivery system is higher than those offered by classical whiteners arbutin and kojic acid, under usual concentrations of use.

### Example 8

A whitening cream gel composition as detailed on Table 2 was prepared, containing the extract of Alpinia officinarum in a delivery system.

TABLE 2
WHITENING CREAM GEL

| Components | % (w/w) |
|---|---|
| Sodium Acrylate/Sodium Acriloyl Dimethyl | 3.50 |
| Taurate, Water, Capric-Caprylic Acid | |
| Triglycerides, Hydrogenated Castor Oil (PEG-40) | |
| Isononyl Isononanoate | 3.00 |
| Miristyl Lactate | 1.00 |
| Water | 86.00 |
| Disodium EDTA | 0.10 |
| Potassium Sorbate | 0.30 |
| Phenoxyethanol | 0.70 |
| Tocopheryl Acetate | 0.10 |
| Extract of Alpinia offinarum in a structured lipid | 3.00 |
| network for delivery of bioactive compounds | |

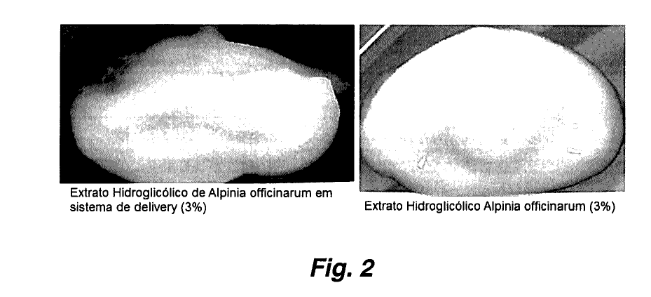

FIG. 2 shows an example of a cosmetic composition of Table 2, containing the extract of Alpinia officinarum in a delivery system, in comparison to the same formulation containing an extract in its free form. We can observe strong colors in the formulation containing the extract of Alpinia officinarum in free form.

### Example 9

Whitening cream compositions were prepared as detailed on Tables 3 and 4, containing the extract of Alpinia officinarum in a delivery system.

TABLE 3
NIGHT WHITENER CREAM

| Components | % (w/w) |
|---|---|
| Sunflower Oil, Polyacrylic acid, Xilityl | 5.00 |
| Sesquicaprilate, Glyceryl Stearate, Candelilla | |
| Wax and Sodium Hydroxide | |
| Sunflower Oil, Corn Oil, Sesame Oil, | 2.00 |
| Macadamia Oil and Olive Oil | |
| Murumuru Butter | 0.50 |
| Dimethicone | 1.00 |
| Water | 86.00 |
| Glycerin | 0.50 |
| Phenoxyethanol, Methylparaben, Ethylparaben, | 0.50 |
| Propylparaben and Butylparaben | |
| Tocopheryl Acetate | 0.10 |
| Extract of Alpinia officinarum in a structured lipid | 3.00 |
| network for delivery of bioactive compounds | |

TABLE 4
DAY WHITENER CREAM

| Components | % (w/w) |
|---|---|
| C20-C22 Alkyl Phosphate, C20-C22 Alcohols | 1.00 |
| Isononyl isononanoate | 3.00 |
| Murumuru Butter | 1.00 |
| Homosalate | 5.00 |
| Octocrylene | 3.00 |
| Bis-Ethyl-Hexoxyphenol Methoxyphenyl | 2.50 |
| Triazine | |
| Water | 0.10 |
| Disodium EDTA | 0.05 |
| Triethanolamine | 0.15 |
| Glycerin | 3.00 |
| Hydroxyethyl Acrylate/Sodium Acriloyl | 2.00 |
| Dimethyl Taurate, Isohexadecane and | |
| Polysorbate 60 | |
| Phenoxyethanol, Butylparaben, Ethylparaben, | 0.50 |
| Propylparaben and Methylparaben | |
| Bis-Benzotriazolyl Tetramethylphenol, Water, | 2.00 |
| Decyl Glucoside, Propylene Glycol and | |
| Xanthan Gum | |
| Extract of Alpinia officinarum in a structured | 3.00 |
| lipid network for delivery of bioactive | |
| compounds | |

### Example 10

### Clinical Safety Assay

The allergenic potential was investigated by means of an assay called HRIPT (Human Repeated Insult Patch Test for delayed contact hypersensitivity). The object of the test is to prove the lack of irritation potential (primary and accumulated skin irritation) and allergy (sensitization) of the product under investigation.

To perform the study 55 Research Subjects were selected—phototypes I to IV, both genders, between 18 and 70 years old, with full skin on the test region. The chosen application area was the dorsal region of the research subjects.

The study was performed in three stages: Primary Skin Irritation, Accumulated Skin Irritation and Skin Sensitization, under maximized conditions, wherein dressings containing the product were applied to the dorsal region of volunteers to prove the lack of irritation potential and allergies. Readings were performed according to the standard reading scale by the International Contact Dermatitis Research Group (ICDRG).

The study observed that cosmetic compositions as mentioned by Example 9 do not present primary skin irritation potential, accumulated skin irritation or skin sensitization.

1. WHITENING ACTIVE INGREDIENT, characterized by comprising:
(a) extract of Alpina officinarum (a-1), and
(b) structured lipid network for delivery of bioactive compounds.

2. WHITENING ACTIVE INGREDIENT, characterized by comprising:
(a) association between extracts of Physalis angulata (a-2), Bidens pilosa (a-3) and Achyrocline satureioides (a-4); and
(b) structured lipid network for delivery of bioactive compounds.

3. WHITENING ACTIVE INGREDIENT of any of claim 1 or 2, characterized by the extract(s) of component (a) being extracted in ethanol, glycols, water or their mixtures.

4. WHITENING ACTIVE INGREDIENT of claim 3, characterized by the extract(s) of component (a) being extract(s) in water and butylene glycol.

5. WHITENING ACTIVE INGREDIENT of any of claim 1 or 2, characterized by the component (b) comprising at least one lipid agent and a hydrocolloid.

6. WHITENING ACTIVE INGREDIENT of claim 5, characterized by the lipid agent being selected from the group consisting of: squalene, lecithin, phosphatidylcholine (originating from soy or egg), cholesterol, L-a-dioleoyl phosphatidylethanolamine, dimethyldioctadecyl ammonium bromide, 1,2-dioleoyl-3-trimethylammonium propane, 1,2-diacyl-3-dimethylammonium propane, dioleoxypropyltrimethylammonium, 2,3-dioleoyloxy-N-[(sperminocarboxamine)ethyl]-N,N-dimethyl-1-propanamine], dioctadecyl dimethylammonium bromide, dim iristoylphosphatidylcholine, distearoylphosphatidylcholine, dilauroylphosphatidylcholine, dipalmitoylphosphatidylcholine, phosphatidylethanolamine, phosphatidylinositol, sphingomyelin, ceramides and linoleic acid.

7. WHITENING ACTIVE INGREDIENT of claim 6, characterized by the lipid agent being soy phosphatidylcholine.

8. WHITENING ACTIVE INGREDIENT of claim 5, characterized by the hydrocolloid being selected from the group consisting of: cellulose and derivatives (hydroxyethylcellulose, carboxymethylcellulose, methylcellulose), gum Arabic, karaya gum, ghatti gum, tragacanth gum, starch and derivatives, pectin (esterified or starched), guar gum, locust bean gum, tara gum, tamarind gum, konjac manana, agar, carrageen, alginate, xanthan gum, curdlan, dextran, gellan gum, gelatin, caseinate, milk serum protein and chitosan.

9. COSMETIC COMPOSITION, characterized by comprising the whitening active ingredient as defined by claim 1 or 2.

10. COSMETIC COMPOSITION of claim 9, characterized by the whitening active ingredient being included within a concentration range between 0.1 and 20.0% (w/v).

11. COSMETIC COMPOSITION of claim 10, characterized by the whitening active ingredient being included within a concentration range between 0.1 and 10.0% (w/v).

12. COSMETIC COMPOSITION of any of claims 9 to 11, characterized by also comprising a UV blocking agent selected from the group consisting of: acrylates (e. g. ethyl 2-cyano 3,3-diphenylacrylate and 2-ethyl-hexyl 2-cyano-3,3-diphenylacrylate); benzophenones (e. g. benzofenone-3 and benzofenone-4); imidazole derivatives (e. g. 2-phenyl benzimidazole 5-sulfonic acid); p-aminobenzoic acid derivatives (e. g. p-aminobenzoic acid and glyceryl p-aminobenzoate); benzotriazole derivatives (e. g. methylene-bis-benzotriazolyl tetramethylbutylphenol) and inorganic pigments such as titanium, zinc, iron and zirconium oxides.

13. COSMETIC COMPOSITION of claim 12, characterized by the UV blocking agent being included in a weight percentage between 0.5 and 10.0%, preferably between 1.0 and 7.0%.

14. COSMETIC COMPOSITION of any of claims 9 to 13, characterized by comprising an emulsifier selected from the group of sorbitol esters, polyglycerol esters and stearates.

15. COSMETIC COMPOSITION of claim 14, characterized by the emulsifier being included in a weight percentage between 0.5 and 15.0%, preferably between 2.0 and 10.0%.

16. COSMETIC COMPOSITION of any of claims 9 to 15, characterized by also comprising an emollient selected from the group of mineral oils, cholesterol, lanolin, silicones, vegetal oils, acids and fatty alcohols.

17. COSMETIC COMPOSITION of claim 16, characterized by the emollient being included in a weight percentage between 0.5 and 10.0%, preferably between 0.5 and 7.0%.

18. COSMETIC COMPOSITION of any of claims 9 to 17, characterized by also comprising a thickener selected from the group of carbomers, vegetal waxes, dioxides, gums and polyacrylamides.

19. COSMETIC COMPOSITION of claim 18, characterized by the thickener being included in a weight percentage between 0.1 and 20.0%, preferably between 1.0 and 7.0%.

20. COSMETIC COMPOSITION of any of claims 9 to 19, characterized by also comprising an antioxidizing agent selected from the group of tocopherol and derivatives, ascorbic acid, BHT and BHA.

21. COSMETIC COMPOSITION of claim 20, characterized by the antioxidant being included in a weight percentage between 1.0 and 5.0%, preferably between 1.0 and 5.0%.

22. COSMETIC COMPOSITION of any of claims 9 to 21, characterized by also comprising an additional whitener agent selected from the group of kojic acid, alfa and beta-arbutin, hydroquinone, linoleic acid, azelaic acid, ferulic acid, ascorbic acid, niacinamide, resveratrol, extracts of Morus alba and extracts of Glycyrrhiza glabra.

23. COSMETIC COMPOSITION of claim 21, characterized by the additional whitening agent being included in a weight percentage between 0.001 and 10%, preferably between 0.1 and 3%.

24. SKIN WHITENING PROCESS, characterized by comprising the topical application of the cosmetic composition as defined by any of claims 9 to 23 over the skin.

25. PROCESS FOR TREATMENT OR PREVENTION OF PIGMENTING DISORDERS, characterized by comprising the topical application of the cosmetic composition as defined by any of claims 9 to 23 over the skin.

26. USE OF A WHITENING ACTIVE INGREDIENT as defined by claim 1 or 2, characterized by being for the manufacture of a composition for skin whitening and/or to avoid or prevent skin pigmentation.

27. USE OF AN ACTIVE WHITENING COMPOSITION as defined by any of claims 9 to 23, characterized by being for skin whitening and/or to avoid or prevent skin pigmentation.

28. PROCESS FOR MANUFACTURING A WHITENING ACTIVE INGREDIENT as defined by claim 1 or 2, characterized by mixing the extract of Alpinia officinarum (a-1) or associating extracts of Physalis angulata (a-2), Bidens pilosa (a-3) and Achyrocline satureioides (a-4) in a structured lipid network, obtained by means of a lipid agent in the presence of an ethanol phase for the delivery of bioactive compounds.

29. ASSOCIATION, characterized by comprising extracts of Physalis angulata, Bidens pilosa and Achyrocline satureioides.
